(19) United States
(12) Patent Application Publication
(10) Pub. No.: US 2024/0307043 A1
(43) Pub. Date: Sep. 19, 2024

(54) DISASSEMBLING ACCESSORY, ACCESSORY BOX AND METHOD FOR DISASSEMBLING PHYSIOLOGICAL SIGNAL MONITORING DEVICE

(71) Applicant: BIONIME CORPORATION, Taichung City (TW)

(72) Inventors: Chun-Mu HUANG, Taichung City (TW); Hsueh-Chuan Chang, Taichung City (TW); Hung-Wen Chiang, Taichung City (TW)

(73) Assignee: BIONIME CORPORATION, Taichung City (TW)

(21) Appl. No.: 18/604,272

(22) Filed: Mar. 13, 2024

Related U.S. Application Data
(60) Provisional application No. 63/490,314, filed on Mar. 15, 2023.

(51) Int. Cl. A61B 17/00

(52) U.S. Cl. CPC A61B 17/00; A61B 2017/00734; A61B 2017/00982

(57) ABSTRACT

A disassembling accessory for disassembling a physiological signal monitoring device includes a base seat having an accommodating space, and a cover body connected to the base seat and movable between covering and open states. The physiological signal monitoring device includes a sensor kit having an through hole, and a transmitter removably mounted to and covering the sensor kit. The cover body has a button being movable and a pushing member connected co-movably to the button. When the cover body is in the covering state, the button is operable to drive the pushing member to move such that the pushing member extends through the through hole to push the transmitter for disassembling the transmitter from the sensor kit.

### CROSS-REFERENCE TO RELATED APPLICATION

This application claims benefit of U.S. Provisional Patent Application No. 63/490,314, filed on Mar. 15, 2023, which is incorporated by reference herein in its entirety.

### FIELD

The disclosure relates to a disassembling accessory, and more particularly to a disassembling accessory for disassembling a physiological signal monitoring device.

### BACKGROUND

Blood glucose control is critical to patients suffering from diabetes or to reducing complications caused thereby. Continuous glucose monitoring (CGM) is a popular method for tracking changes in glucose levels by taking glucose measurements of an individual at regular intervals.

A CGM system is mounted on a host, i.e., a patient suffering from diabetes, and usually includes a biosensor inserted beneath the host's skin for measuring a physiological signal corresponding to the blood glucose concentration level of the host, a transmitter for transceiving the physiological signal, and a mounting seat for interconnecting the biosensor and the transmitter and mounting on the host. Due to such intrusive nature of the biosensor, the host's body may be hypersensitive to the biosensor and in turn develops a severe allergic reaction, so the biosensor needs to be replaced on a weekly or bi-weekly basis. Given that the transmitter of the CGM system is more expensive than the biosensor and the mounting seat, frequent replacement of these electric components may also cause environmental waste.

### SUMMARY

Therefore, an object of the disclosure is to provide a disassembling accessory for disassembling a transmitter of a physiological signal monitoring device.

According to an aspect of the disclosure, a disassembling accessory adapted for disassembling a physiological signal monitoring device is provided. The physiological signal monitoring device includes a bottom seat that has at least one through hole, a sensor that is mounted to the bottom seat, and a transmitter that is removably mounted to and covering the bottom seat. The disassembling accessory includes a base seat and a cover body. The base seat has an accommodating space. The cover body is connected to one side of the base seat, and is operable to move relative to the base seat between a covering state and an open state. The cover body has a button, and at least one pushing member linked to the button. When the cover body is in the open state, the accommodating space is adapted to receive the physiological signal monitoring device therein with the transmitter facing the accommodating space. When the cover body is in the covering state, the button is operable to drive the at least one pushing member to move in a first direction such that the at least one pushing member is adapted to extend through the at least one through hole to push the transmitter for disassembling the transmitter from the bottom seat.

According to another aspect of the disclosure, a disassembling accessory adapted for disassembling a physiological signal monitoring device is provided. The physiological signal monitoring device includes a sensor kit that has at least one through hole at a bottom portion thereof, and a transmitter that is removably coupled to the sensor kit. The disassembling accessory includes a base seat and a cover body. The base seat has an accommodating space and is adapted for accommodating the physiological signal monitoring device. The cover body is connected to one side of the base seat, and is operable to move relative to the base seat between a covering state and an open state. The cover body has at least one pushing member. When the cover body is in the open state, the accommodating space is adapted to receive the physiological signal monitoring device therein with the transmitter facing the accommodating space. When the cover body is in the covering state, the at least one pushing member is operable to move in a first direction such that the at least one pushing member is adapted to extend through the at least one through hole to push the transmitter for disassembling the transmitter from the sensor kit.

According to yet another aspect of the disclosure, an accessory box adapted for disassembling a physiological signal monitoring device is provided. The physiological signal monitoring device includes a sensor kit that has at least one through hole at a bottom portion thereof, and a transmitter removably coupled to the sensor kit. The accessory box includes a base seat and a cover body. The base seat has a disassembling unit having an accommodating space and a charging unit disposed adjacent to the disassembling unit and formed with a charging slot. The cover body is connected to one side of the base seat, and is operable to move relative to the base seat between a covering state and an open state. The cover body has at least one pushing member disposed adjacent to the disassembling accessory when the cover body is in the covering state. When the cover body is in the open state, the accommodating space is adapted to receive the physiological signal monitoring device therein with the transmitter facing the accommodating space. When the cover body is in the covering state, the at least one pushing member is operable to move in a first direction such that the at least one pushing member is adapted to extend through the at least one through hole to push the transmitter for disassembling the transmitter from the sensor kit. The charging slot of the charging unit is adapted to receive the transmitter that is disassembled from the sensor kit for charging a battery of the transmitter.

According to still another aspect of the disclosure, a method for disassembling a physiological signal monitoring device is provided. The physiological signal monitoring device includes a sensor kit that is formed with at least one opening at a bottom portion thereof, and a transmitter that is removably mounted to and covering the sensor kit. The method includes the steps of:
providing a disassembling accessory that includes
   a base seat having an accommodating space for receiving the physiological signal monitoring device therein, and
   a cover body connected to one side of the base seat, movable relative to the base seat between a covering state and an open state, where the cover body covers and uncovers the base seat, respectively, and including at least one pushing member;
uncovering the cover body so that the cover body is in the open state;
placing the physiological signal monitoring device into the accommodating space with the transmitter facing a bottom of the accommodating space;
covering the cover body so that the cover body is in the covering state;
disassembling the transmitter from the sensor kit by driving the at least one pushing member to move in a first direction, to extend through the at least one opening, and to push the transmitter; and
retrieving the transmitter by removing the transmitter from the base seat.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is an exploded perspective view of a physiological signal monitoring device to be disassembled by a disassembling accessory of the present disclosure.

FIG. 2 is a sectional view of the physiological signal monitoring device.

FIG. 3 is a perspective view of a disassembling accessory of a first embodiment according to the present disclosure.

FIG. 4 is a partly exploded perspective view of the disassembling accessory of the first embodiment disposed in an open state.

FIG. 5 is an exploded perspective view of the disassembling accessory of the first embodiment

FIG. 6 is a perspective view of the physiological signal monitoring device being disposed in the disassembling accessory of the first embodiment when a cover of the disassembling accessory is in the open state.

FIG. 7 is a sectional view of the disassembling accessory of the first embodiment of FIG. 6.

FIG. 8 is a sectional view of the disassembling accessory of the first embodiment.

FIG. 9 is a sectional view taken along the line IX-IX in FIG. 8.

FIG. 10 is a sectional view similar to FIG. 9, but illustrating a button of the disassembling accessory of the first embodiment being pressed to disassemble a transmitter from a sensor kit of the physiological signal monitoring device.

FIG. 11 is a sectional view similar to FIG. 10, but illustrating the transmitter being disengaged from the sensor kit.

FIG. 12 is a sectional view similar to FIG. 11, but illustrating disassembling of the transmitter being completed.

FIGS. 13(a) to 13(d) are perspective views of the disassembling accessory of the first embodiment, illustrating the physiological signal monitoring device being disposed in the disassembling accessory, being disassembled, and being removed from the disassembling accessory.

FIG. 14 is sectional view of a modification of the disassembling accessory of the first embodiment.

FIG. 15 is a perspective view of a disassembling accessory of a second embodiment according to the present disclosure.

FIG. 16 is a sectional view of the disassembling accessory of the second embodiment.

FIG. 17 is a sectional view of a modification of the disassembling accessory of the second embodiment.

FIG. 18 is a sectional view of a disassembling accessory of a third embodiment according to the present disclosure.

FIGS. 19(a) to 19(d) are perspective views of the disassembling accessory of the third embodiment, illustrating the physiological signal monitoring device being disposed in the disassembling accessory, being disassembled, and being removed from the disassembling accessory.

FIG. 20 is a perspective view of a disassembling accessory of a fourth embodiment according to the present disclosure.

FIG. 21 is a sectional view taken along the line XXI-XXI in FIG. 20.

FIG. 22 is an accessory box of a first embodiment according to the present disclosure for disassembling a physiological signal monitoring device.

FIG. 23 is a schematic perspective view of the accessory box of the first embodiment disposed in an open state.

FIG. 24 is a sectional view of the accessory box of the first embodiment.

FIG. 25 is a perspective view of the accessory box of a second embodiment according to the present disclosure.

FIG. 26 is a sectional view of an accessory box of a third embodiment according to the present disclosure.

FIG. 27 is a flow chart of a method for disassembling the physiological signal monitoring device to be implemented by the present disclosure.

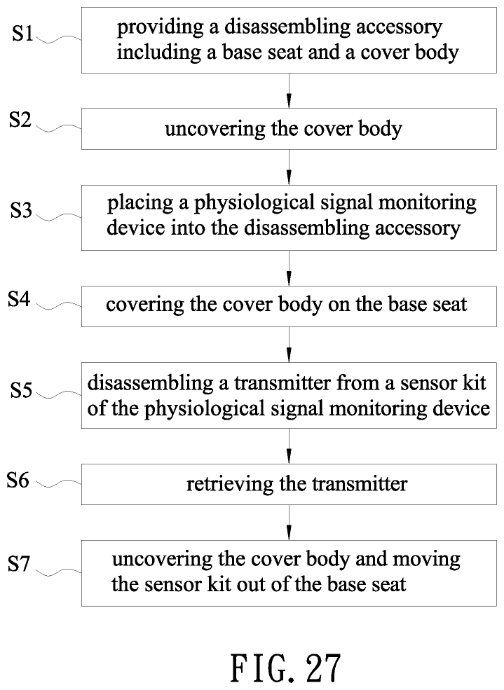

### DETAILED DESCRIPTION

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

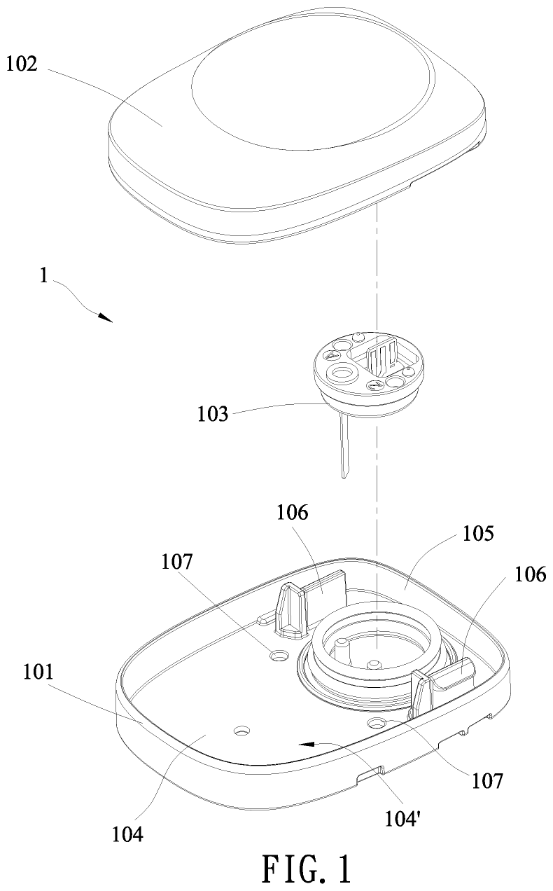

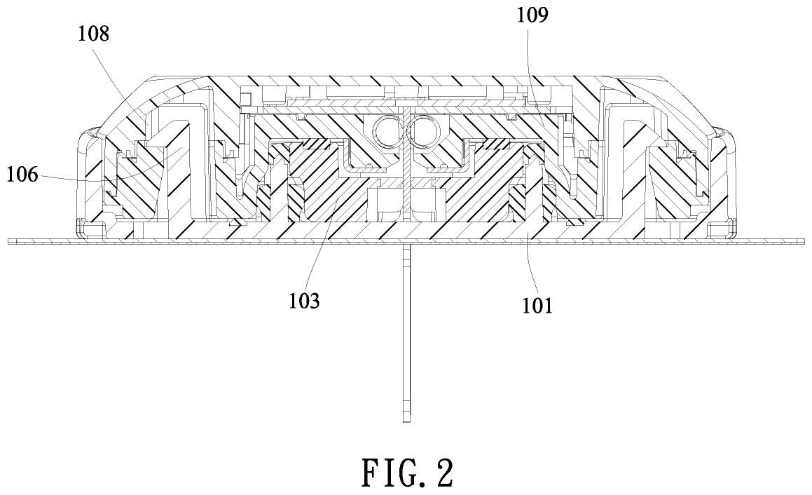

Referring to FIGS. 1 and 2, a physiological signal monitoring device 1 is shown. The physiological signal monitoring device 1 includes a bottom seat 101, a sensor 103 mounted to the bottom seat 101, and a transmitter 102 removably coupled to and covers on the bottom seat 101. The bottom seat 101 has a bottom plate 104 having a top surface 104′, a surrounding wall 105 extending upwardly from a periphery of the bottom plate 104, two first coupling structures 106 disposed on and protruding upwardly from the top surface 104′, and two through holes 107 formed through the bottom plate 104 and disposed respectively adjacent to the first coupling structures 106. The bottom plate 104 is disposed to be mounted on a skin surface of a host. The sensor 103 is for sensing an analyte of a host and for transmitting a physiological signal corresponding to the blood glucose concentration level of the host to the transmitter 102. The transmitter 102 is electrically connected to the sensor 103 for transceiving the physiological signal when covering on the bottom seat 101. In one embodiment, the bottom seat 101 and the sensor 103 are integrated as a sensor kit that is disposable, whereas the transmitter 102 may be reused.

Specifically, the transmitter 102 includes a bottom casing 109 and two second coupling structures 108. The second coupling structures 108 are disposed on the bottom casing 109. When the transmitter 102 covers the bottom seat 101, the second coupling structures 108 respectively and removably engage the first coupling structures 106 with the bottom casing 109 facing the top surface 104′ of the bottom plate 104. However, the quantities of the first coupling structures 106 and the second coupling structures 108 are not limited to any one of the embodiments of the present disclosure.

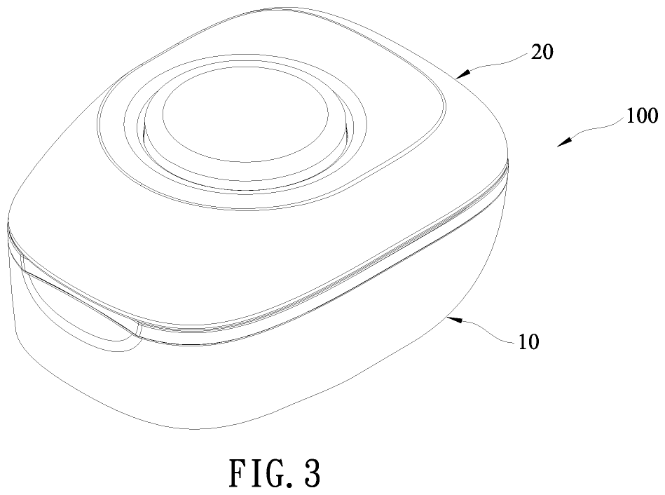

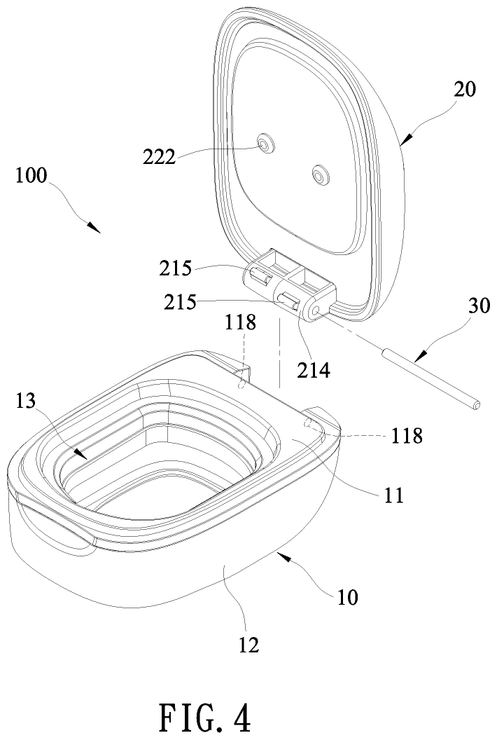

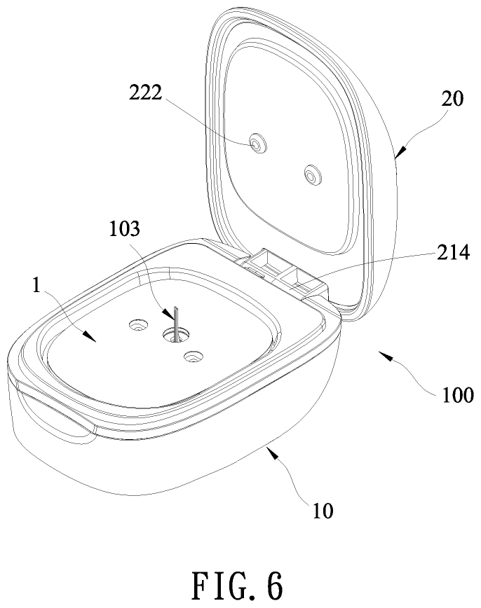

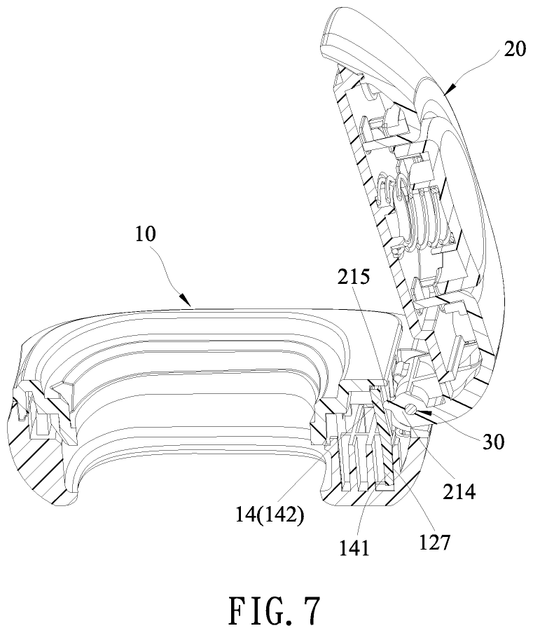

Referring to FIGS. 3 to 9, a dissembling accessory 100 of a first embodiment according to the present disclosure is shown. The dissembling accessory 100 is adapted for disassembling the physiological signal monitoring device 1 and includes a base seat 10 and a cover body 20. The base seat 10 has an accommodating space 13. The cover body 20 is connected to one side of the base seat 10, and is operable to move relative to the base seat 10 between a covering state (see FIG. 3) and an open state (see FIG. 6), where the cover body 20 covers and uncovers the base seat 10, respectively. The cover body 20 includes a button 23 and at least one pushing member 24 linked to and driven by the button 23 to move when the cover body 20 is in the cover state. As shown in FIGS. 6 and 7, when the cover body 20 is in the open state, the physiological signal monitoring device 1 is disposed in the accommodating space 13 with the transmitter 102 facing the accommodating space 13. Specifically, the physiological signal monitoring device 1 is disposed in the accommodating space 13 with the transmitter 102 facing a bottom of the accommodating space 13. In this embodiment, the disassembling accessory 100 includes a pivot shaft 30 that pivotably connects the cover body 20 to the base seat 10.

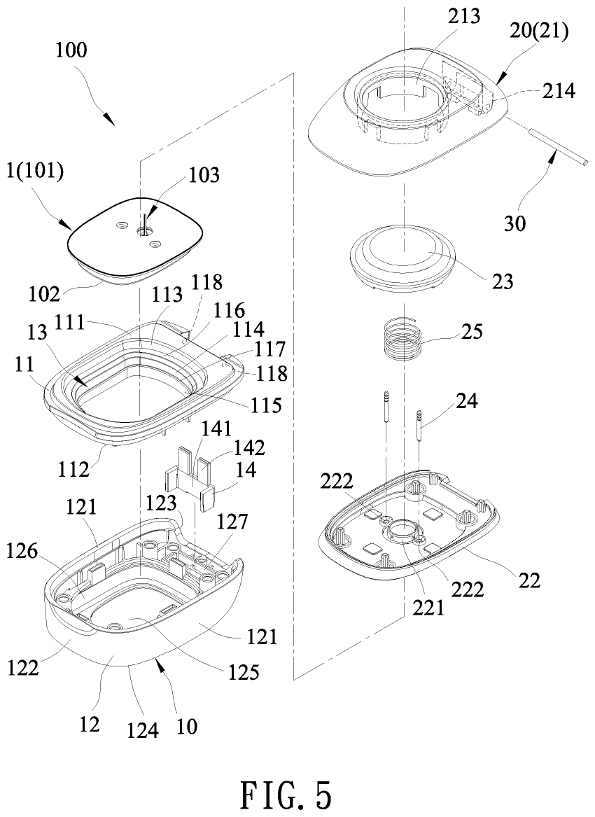

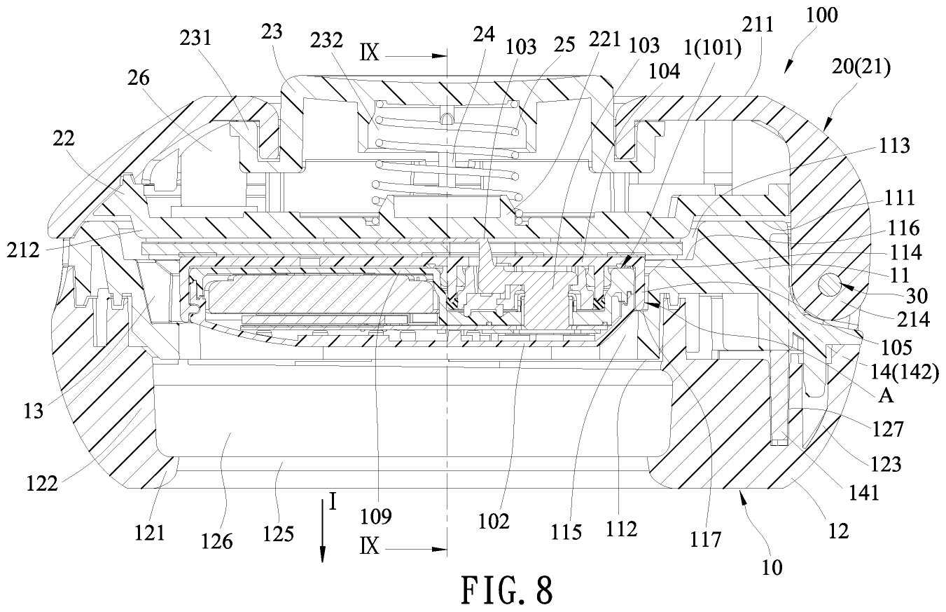

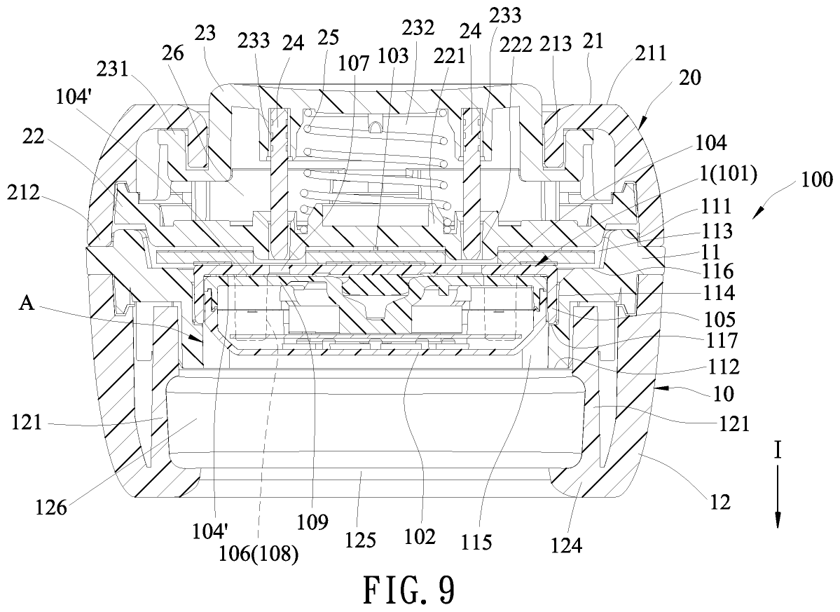

As shown in FIGS. 8 and 9, a sectional view of the disassembling accessory 100 of the first embodiment is shown. The base seat 10 includes an upper housing 11 and a lower housing 12 that is connected to the upper housing 11 and that cooperates with the upper housing 11 to define the accommodating space 13. As shown in FIG. 5, the upper housing 11 is an annular frame and has an upper surface 111, a lower surface 112 opposite to the upper surface 111, a recess portion 113 formed in the upper surface 111, a first receiving space 114 formed at a position opposite to the recess portion 113, and a through hole 115 formed in the lower surface 112. The upper housing 11 further has an upper shoulder surface 116 formed between the recess portion 113 and the first receiving space 114, a lower shoulder surface 117 formed between the first receiving space 114 and the lower surface 112, and a pair of pivot connecting holes 118 formed at a rear portion of the upper housing 11.

The lower housing 12 includes a pair of side walls 121, a front wall 122, a rear wall 123, and a bottom wall 124. The side walls 121 are spaced apart from each other. The front wall 122 is connected to the side walls 121. The rear wall 123 is opposite to the front wall 122 and is connected to the side walls 121. The bottom wall 124 is formed with a first opening 125 and is connected to the side walls 121, the front wall 122, and the rear wall 123. The side walls 121, the front wall 122, the rear wall 123, and the bottom wall 124 cooperate with one another to define a second receiving space 126 that is in spatial communication with the first receiving space 114. Specifically, the accommodating space 13 has the first receiving space 114 and the second receiving space 126. The lower shoulder surface 117, the through hole 115, and the bottom surface 112 cooperate with one another to define a limiting structure (A). It should be noted that the limiting structure (A) is formed in the first receiving space 114 and is for limiting the physiological signal monitoring device 1 therein when the physiological signal monitoring device 1 is disposed in the accommodating space 13 while facing the accommodating space 13.

The second receiving space 126 is in spatial communication with external environment via the first opening 125. As depicted in FIG. 8, the first opening 125 has an opening size that is adapted to be greater than a maximum cross sectional area of the transmitter 102 or that is not smaller than a bottom wall area of the bottom casing 109 of the transmitter 102. The first receiving space 114 and the second receiving space 126 are arranged in a first direction (I) and are proximate to each other.

As shown in FIGS. 5 and 8 to 9, the cover body 20 includes an upper lid 21 and a lower lid 22 connected to the upper lid 21 and cooperating with the upper lid 21 to define an inner space 26. The button 23 is mounted on the upper lid 21. It should be noted that the number of the at least one pushing member 24 is two in this embodiment and the pushing members 24 are driven by the button 23 to move. In this embodiment, the cover body 20 further includes a biasing member 25 disposed in the inner space 26, disposed between and abutting against the lower lid 22 and the button 23, and biasing the button 23 away from the lower lid 22.

The upper lid 21 is formed with a central hole 213 for mounting the button 23 therein, and has an upper portion 211, a lower portion 212 opposite to the upper portion 211, and a lug portion 214 formed at a rear of the upper lid 21. The pivot shaft 30 extends through the lug portion 214 and the pivot connecting holes 118 to pivotably connect the cover body 20 and the base seat 10.

The lower lid 22 includes a positioning ring 221 aligned with the central hole 213 in the first direction (I), and is formed with two sliding holes 222 that are disposed at two opposite sides of the positioning ring 221.

The button 23 is mounted in the central hole 213 of the upper lid 21, is movable relative to the upper lid 21, includes a rim portion 231 disposed in the inner space 26 and engaging the upper lid 21, and is formed with a positioning hole 232 aligned with the positioning ring 221 in the first direction (I), and two insertion holes 233 aligned respectively with the sliding holes 222 and respectively receiving the pushing members 24 therein. That is to say, each of the pushing members 24 extends into the respective one of the insertion holes 233 of the button 23 and one of the sliding holes 222 that is aligned with the respective one of the insertion holes 233.

The pushing members 24 are disposed in the inner space 26 and are driven by the button 23 to move in the first direction (I) and extend outwardly of the lower lid 22. In this embodiment, the pushing members 24 are configured as two pillars, and extend through and are slidable along the sliding holes 222, respectively.

The biasing member 25 provides a resilient force for biasing the button 23 in a direction away from the lower lid 22. In this embodiment, the biasing member 25 is a compression spring, has two opposite ends abutting respectively against the lower lid 22 and the button 23, is disposed in the positioning hole 232, and surrounds the positioning ring 221. That is to say, the biasing member 25 is disposed between the positioning ring 221 and the positioning hole 232.

As shown in FIGS. 5 and 7, the disassembling accessory 100 further includes a rotation limiting member 14 mounted to the base seat 10 at a position adjacent to the pivot shaft 30 and disposed for positioning the cover body 20 relative to the base seat 10 in the open state. Specifically, the lower housing 12 is formed with a slot 127 at the rear wall 123. The rotational member 14 includes a positioning segment 141 and a blocking segment 142 connected to the positioning segment 141 and extending toward the upper housing 11. The upper lid 21 further includes an abutment block 215 (see FIG. 7) disposed adjacent to the lug portion 214 and the rotational member 14. When the cover body 20 is in the open state, the abutment block 215 abuts against the blocking segment 142, which thus prevents unintentional movement of the cover body 20 from the open state to the covering state.

A method for dissembling the physiological signal monitoring device 1 implemented by the disassemble accessory 100 is described in the following.

FIG. 3 illustrates the cover body 20 being in the covering state.

The method includes the following steps S1 to S5.

In step S1, the dissembling accessory 100 that is shown in FIGS. 1 to 9 and that includes the base seat 10 and the cover body 20 described above is provided.

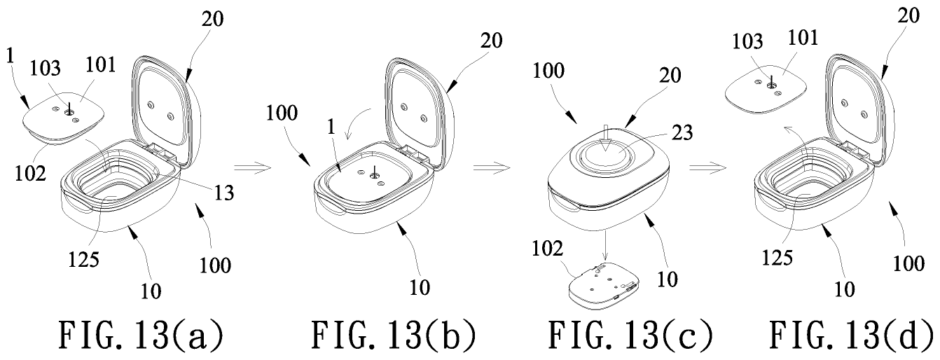

In step S2, as shown in FIGS. 6 and 13(a), the cover body 20 is uncovered (i.e., in the open state).

In step S3, as shown in FIGS. 6 and 13(b), the physiological signal monitoring device 1 is placed into the accommodating space 13 with the transmitter 102 facing the bottom of the accommodating space 13. Further referring to FIGS. 8 and 9, the limiting structure (A) limits the sensor kit of the physiological signal monitoring device 1. Specifically, the surrounding wall 105 of the bottom seat 101 is disposed on and limited by the limiting structure (A), so the physiological signal monitoring device 1 is received in a portion of the accommodating space 13, i.e., the first receiving space 114. At this time, the transmitter 102 of the physiological signal monitoring device 1 is proximate to another portion of the accommodating space 13, i.e., the second receiving space 126 such that, upon a step of dissembling the transmitter 102 (referring to the step S5) from the sensor kit, the transmitter 102 is moved to the another portion of the accommodating space 13, i.e., the second receiving space 126.

In step S4, as shown in FIGS. 8 and 9, the cover body 20 covers the base seat 10 (i.e., in the covering state). It should be noted that, as depicted in FIG. 8, the sensor 103 has a portion that is bent when being covered by the cover body 20. When the cover body 20 is in the covering state, the button 23 is operable to drive the pushing members 24 to move in the first direction (1) such that the pushing members 24 are adapted to respectively extend through the through holes 107 to push the transmitter 102 for disassembling the transmitter 102 from the bottom seat 101. Specifically, the button 23 is operated in the first direction (I) and thus the pushing members 24 are moved in the first direction (I) respectively through the through holes 107, but the present disclosure is not limited in this respect.

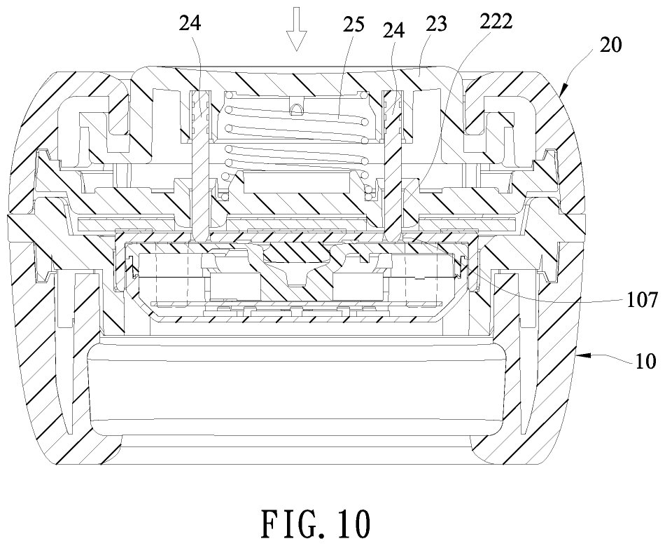

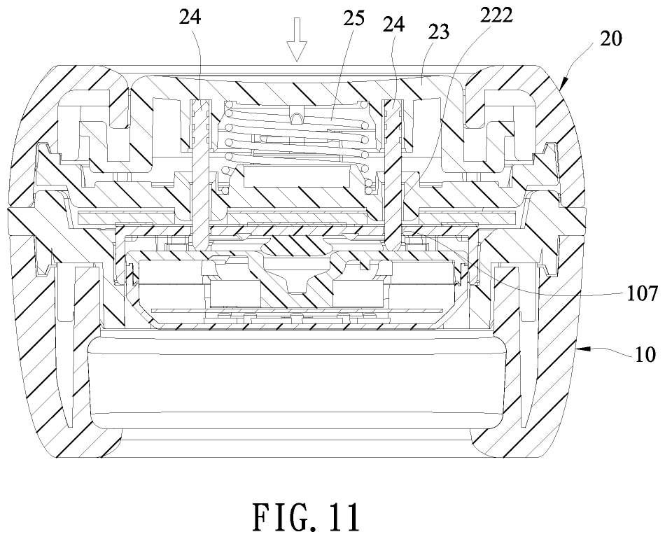

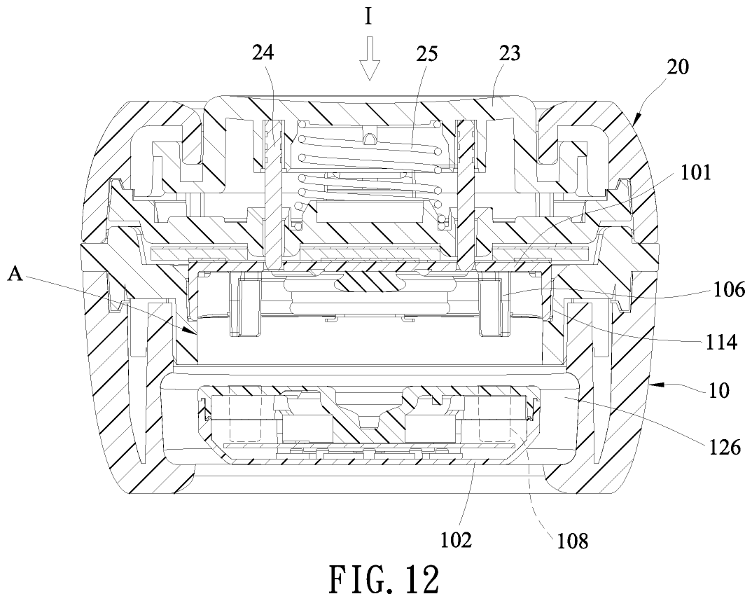

In step S5, as shown in FIGS. 10 to 12, and 13(c), the transmitter 102 is disassembled from the sensor kit. Specifically, the button 23 is pushed in the first direction (I) to move the pushing members 24 in the first direction (I) to respectively extend through the through holes 107 and to push the transmitter 102. At this time, the biasing member 25 is compressed to store a restoring force. Consequently, as shown in FIG. 12, the second coupling structures 108 disengage respectively from the first coupling structures 106 to thereby disassembling the transmitter 102 from the bottom seat 101. It should be noted that the transmitter 102 falls out of the first receiving space 114 and into the second receiving space 126 while the bottom seat 101 is retained on the limiting structure (A). After the button 23 is released, the restoring force provided by the biasing member 25 biases the button 23 away from the lower lid 22 to its original position.

In step S6, the transmitter 102 is retrieved to be reused. As shown in FIG. 13(c), the transmitter 102 falls out of the base seat 10 through the first opening 125.

After the transmitter 102 is disassembled from the base seat 10, in step S7, the cover body 20 is uncovered (i.e., in the open state) and the sensor kit, i.e., the bottom seat 101 and the sensor 103, is movable out of the base seat 10 by an external force that is applied via the first opening 125.

Thus, when the sensor 103 is to be replaced, the transmitter 102 may be easily dissembled from the bottom seat 101 and may be reused. Since the transmitter 102 is more expansive than the bottom seat 101 and the sensor 103, the disassembling accessory 100 of the present disclosure lowers the cost for the host. Additionally, the present disclosure is beneficial to the environment, and corporate governance for social responsibility is achieved.

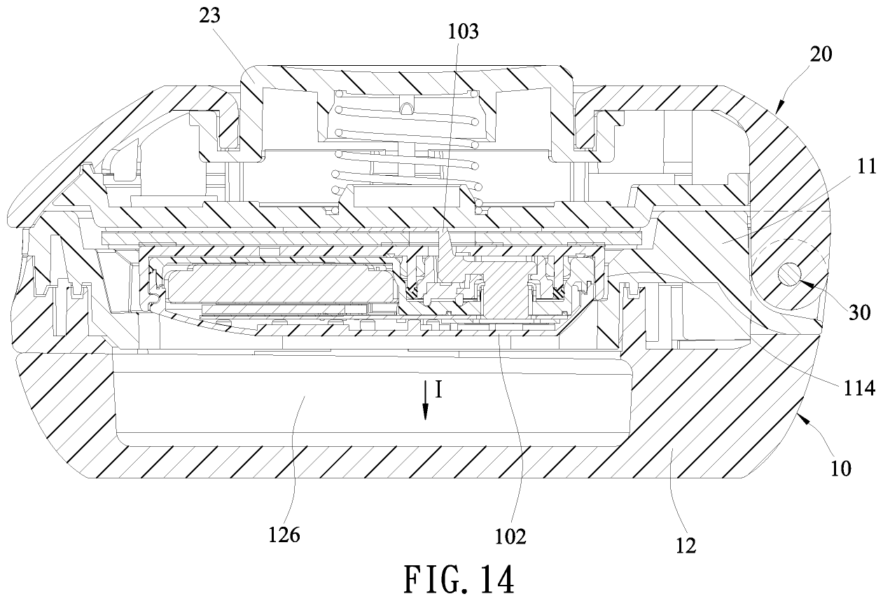

As shown in FIG. 14, a modification of the disassembling accessory 100 of the first embodiment is shown. In this modification, the base seat 10 includes an upper housing 11 defining the first receiving space 114, and a lower housing 12 defining the second receiving space 126 and connected pivotably to the upper housing 11 via the pivot shaft 30. The lower housing 12 is pivotable relative to the upper housing 11 from a limiting state to a non-limiting state, where removal of the transmitter 102 from the second receiving space 126 is permitted. The second receiving space 126 may be configured to temporarily store the transmitter 102 being disassembled. Furthermore, the first opening 125 of the first embodiment may be omitted.

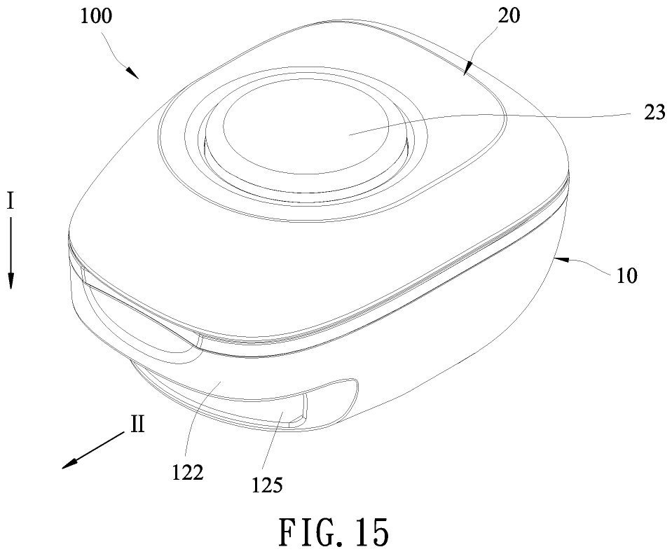

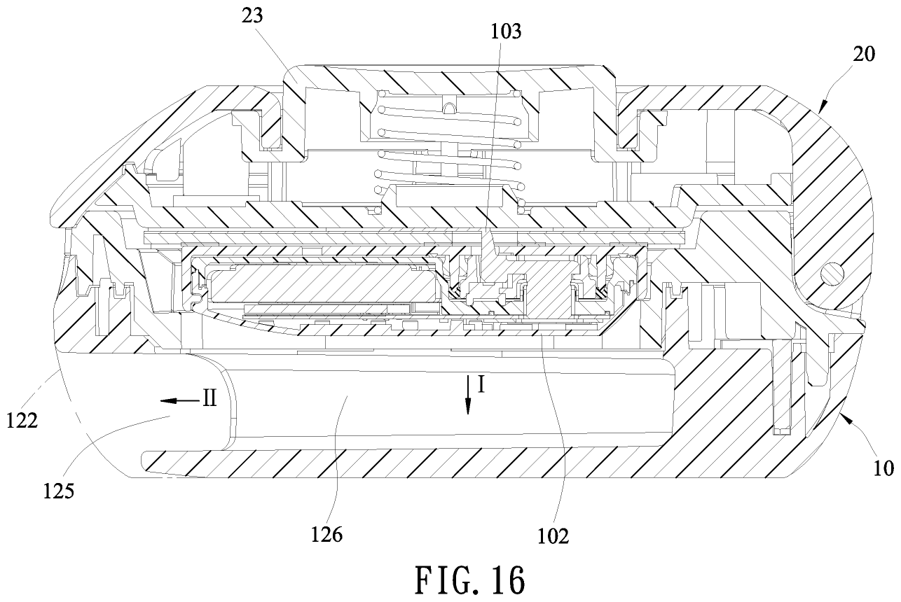

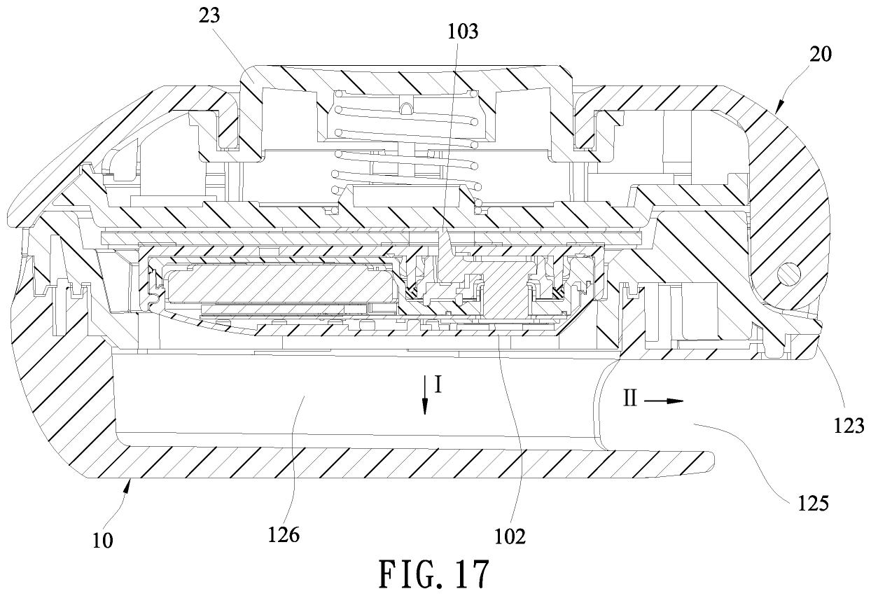

Referring to FIGS. 15 and 16, a disassembling accessory 100 of a second embodiment according to the present disclosure is shown. The second embodiment is similar to the first embodiment and also includes a base seat 10 and a cover body 20. The differences between the disassembling accessories 100 of the first embodiment and the second embodiment reside in the following. In the disassembling accessory 100 of the second embodiment, the first opening 125 is formed in the front wall 122 and is in spatial communication with the second receiving space 126. When the cover body 20 is in the covering state, the button 23 is operated to disassemble the transmitter 102 from the bottom seat 101 so that the transmitter 102 enters the second receiving space 126. Subsequently, the first opening 125 is adapted for passage of the transmitter 102 to exit from the second receiving space 126 to the external environment. In this way, the transmitter 102 may be removed outwardly of the base seat 10 via the first opening 125 in a second direction (II) transverse to the first direction (I) by simply tilting the disassembling accessory 100. Thus, the transmitter 102 may be easily retrieved, and accidental fall of the transmitter 102 upon disassembling of the transmitter 102 from the bottom seat 101 may be prevented. As shown in FIG. 17, a modification of the disassembling accessory 100 of the second embodiment is shown. In this modification, the first opening 125 is formed in the rear wall 123 and is in spatial communication with the second receiving space 126.

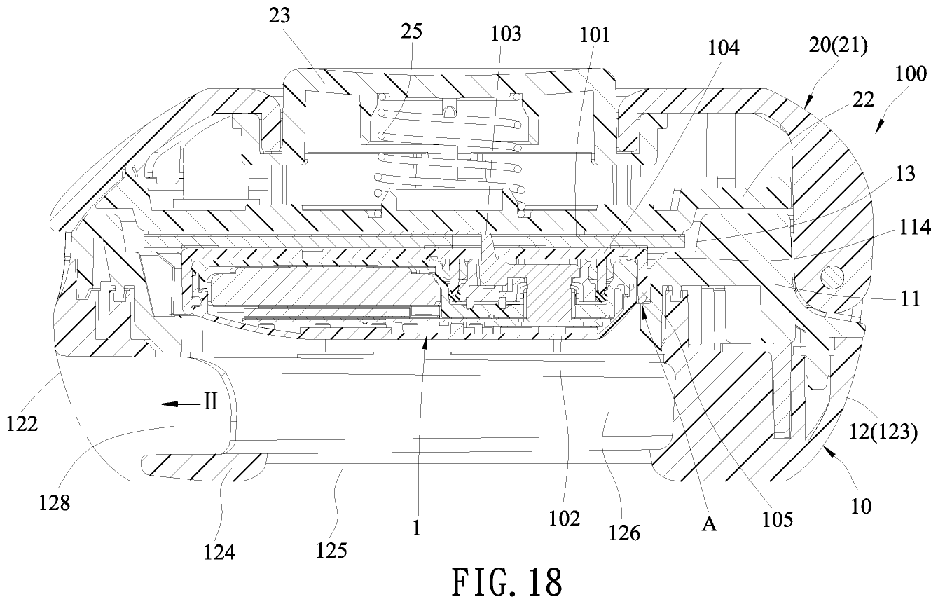

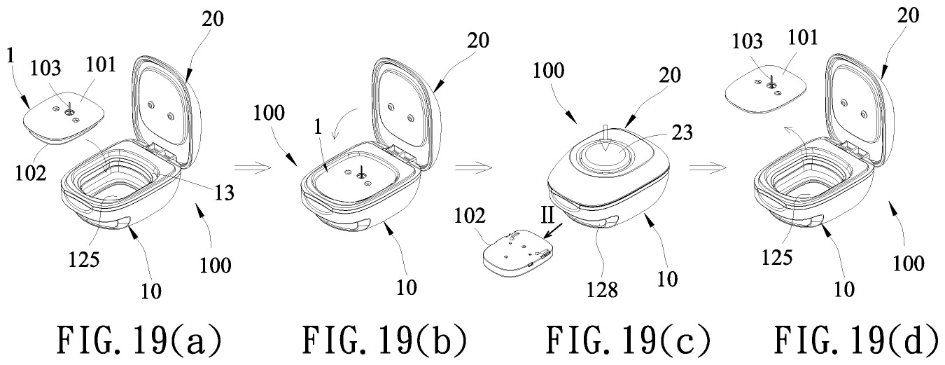

Referring to FIGS. 18 and 19, a disassembling accessory 100 of a third embodiment according to the present disclosure is similar to the disassembling accessory 100 of the first embodiment and also includes a base seat 10 and a cover body 20. It should be noted that the pushing members 24 are not visible in FIG. 16, and it may be appreciated that the pushing members 24 are shown in a sectional view similar to that shown in FIG. 9. The differences between the disassembling accessories 100 of the first embodiment and the third embodiment reside in the following. The base seat 10 of the disassembling accessory 100 of the third embodiment further has a second opening 128 that is formed in the front wall 122 and that is in spatial communication with the second receiving space 126 and the external environment, and the first opening 125 is formed in the bottom wall 124. In this embodiment, dimension of the first opening 125 is smaller than a maximum sectional area of the transmitter 102. The second opening 128 is adapted for passage of the transmitter 102 therethrough to exit from the second receiving space 126 to the external environment when the transmitter 102 is dissembled from the bottom seat 101. In this way, the transmitter 102 may be removed outwardly of the base seat 10 in the second direction (II) by simply tilting the disassembling accessory 100 to thereby retrieve and reuse the transmitter 102. After the transmitter 102 is dissembled from the bottom set 101, falls into the second receiving space 126, and exits from the second receiving space 126 via the second opening 128, the cover body 20 is uncovered (i.e., in the open state) and the bottom seat 101 and the sensor 103 are movable out of the base seat 10 by an external force, e.g., a user's finger, that is applied via the first opening 125. In this way, the sensor kit, i.e., the bottom seat 101 and the sensor 103, may be moved out of the base seat 10 via the first opening 125 and for replacement.

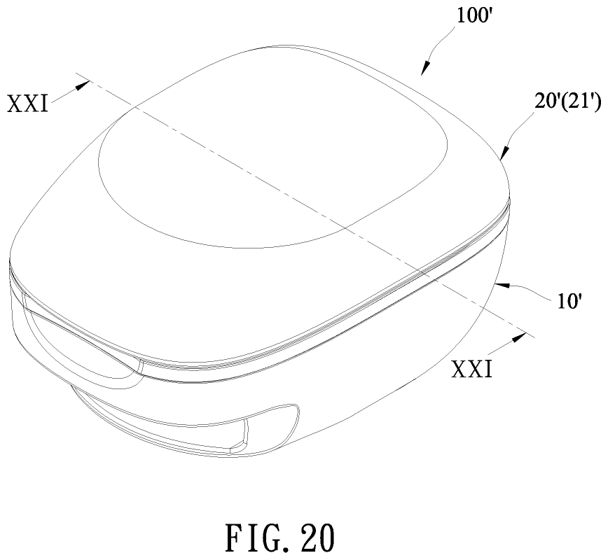

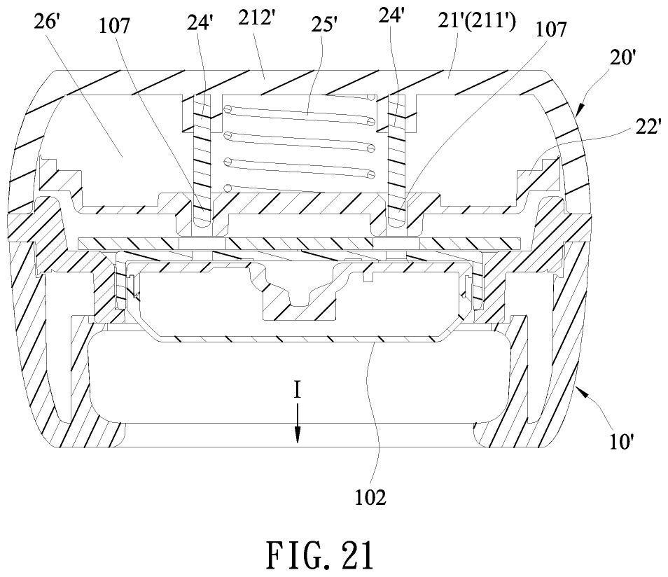

Referring to FIGS. 20 and 21, a disassembling accessory 100′ of a fourth embodiment according to the present disclosure is shown. FIG. 20 is a perspective view of the disassembling accessory 100′ and FIG. 21 is a sectional view of taken along the line XXI-XXI of FIG. 20. The fourth embodiment also includes a base seat 10′ and a cover body 20′.

As shown in FIG. 20, the differences between the disassembling accessories 100, 100′ of the first embodiment and the fourth embodiment reside in that the cover body 20′ of the fourth embodiment does not include the button 23 shown in FIG. 15. Specifically, in this embodiment, the cover body 20′ includes an upper lid 21′, a lower lid 22′ connected to the upper lid 21′, a pair of pushing members 24′, and a biasing member 25′. The upper lid 21′ has at least one soft portion 212″ that is resiliently deformable and that is made of, e.g., silicon through a two-material injection molding process. The soft portion 212″ is connected to and operable to drive the pushing members 24′ to move in the first direction (I) such that the pushing members 24′ are adapted to extend respectively through the through hole 107 to push the transmitter 102 for disassembling the transmitter 102 from the sensor kit.

It should be noted that the biasing member 25′ is disposed in the inner space 26′, and is disposed between and abutting against the lower lid 22′ and the upper lid 21′ for restoring the soft portion 212′. Additionally, the biasing member 25′ facilitates the pushing members 24′ to stably extend through the through holes 107, respectively. In a case where the number of each of the through hole 107 and the pushing member 24′ is only one and the pushing member 24′ is disposed at a center of the upper lid 21′, the biasing member 25′ surrounds the pushing member 24′ to enable stable movement of the pushing member 24 in the first direction (I) through the through hole 107.

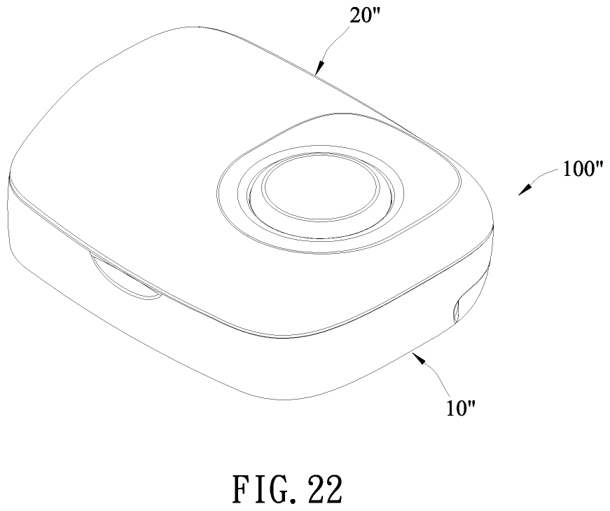

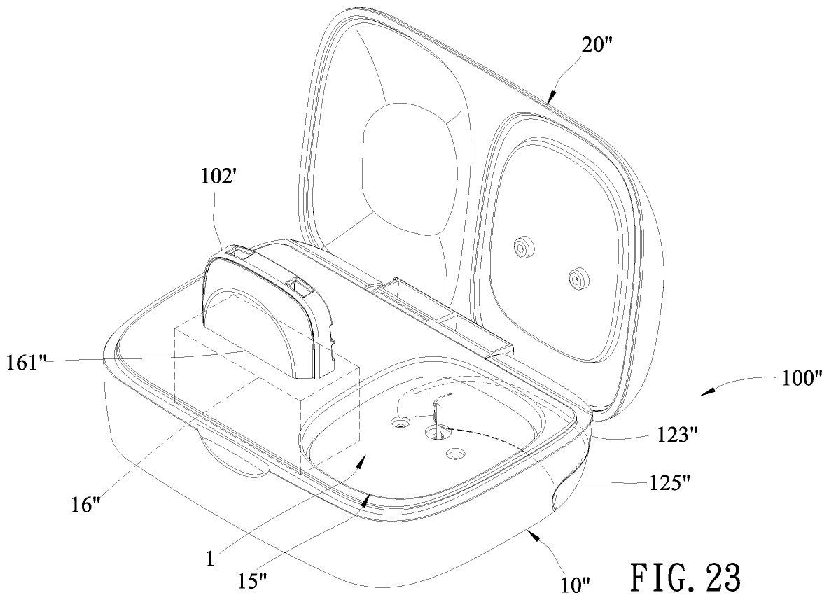

Referring to FIGS. 22 to 24, an accessory box 100″ of a first embodiment according to the present disclosure includes a base seat 10″ and a cover body 20″ pivotably connected to one side of the base seat 10″.

The base seat 10″ has a disassembling unit 15″ that has an accommodating space 13″, and a charging unit 16″ disposed adjacent to the dissembling unit 15″ and that is formed with a charging slot 161″. The accommodating space 13″ has a first receiving space 114″ and a second receiving space 126″ in spatial communication with the first receiving space 114″. The charging slot 161″ is adapted to receive the transmitter 102 that is disassembled from the sensor kit for charging a battery of the transmitter 102. The first opening 125″ is formed in the rear wall 123″ and is in spatial communication with the second receiving space 126″ and the external environment. The difference between the accessory box 100″ and the disassembling accessories 100, 100′ of previous embodiments merely resides in that the accessory box 100″ further includes the charging unit 16″ and the remaining features are substantially the same as those described above and are thus omitted for the sake of brevity.

When the cover body 20″ is in the open state, the physiological signal monitoring device 1 that is to be disassembled is placed in the accommodating space 13″, and the transmitter 102′ of a spare one of the physiological signal monitoring device 1 that is disassembled from the sensor kit of the spare one of the physiological signal monitoring device 1 is inserted in the charging slot 161″ such that a battery of the transmitter 102′ may be charged. In addition, the charging unit 16″ may include a charging port electrically connected to an external power source via a wire for charging the battery of the transmitter 102′. It should be noted that the charging port may be a wireless charging port electrically and wirelessly connected to the external power source.

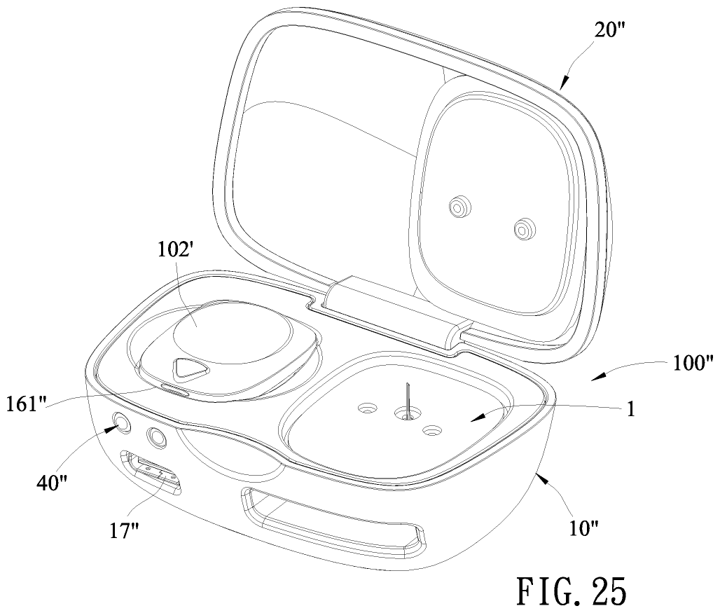

Referring to FIG. 25, an accessory box 100″ of a second embodiment according to the present disclosure is similar to the accessory box 100″ of the first embodiment and the difference therebetween resides in that the charging slot 161″ of the accessory box 100″ of the second embodiment has a flat configuration with a greater opening area than that of the charging slot 161″ of the accessory box 100″ of the first embodiment. Additionally, in this embodiment, the charging unit 16″ further includes a charging port 17″, e.g., type C, that is electrically connected to an external power source via a wire for charging the battery of the transmitter and a charging terminal (not shown) that is electrically connected to the charging port 17″ and the transmitter 102′ for charging the battery of the transmitter 102′. It can be appreciated that a wireless charging technique may be employed in other embodiments of this disclosure and the present disclosure is not limited in this respect.

Furthermore, the accessory box 100″ of the second embodiment further includes an indicating unit 40″ that is disposed on an outer surface of the base seat 10″, that is electrically connected to the charging unit 16″, and that is configured to indicate a charging progress of the transmitter 102. The indicator unit 40″ may include a light emitting diode (LED).

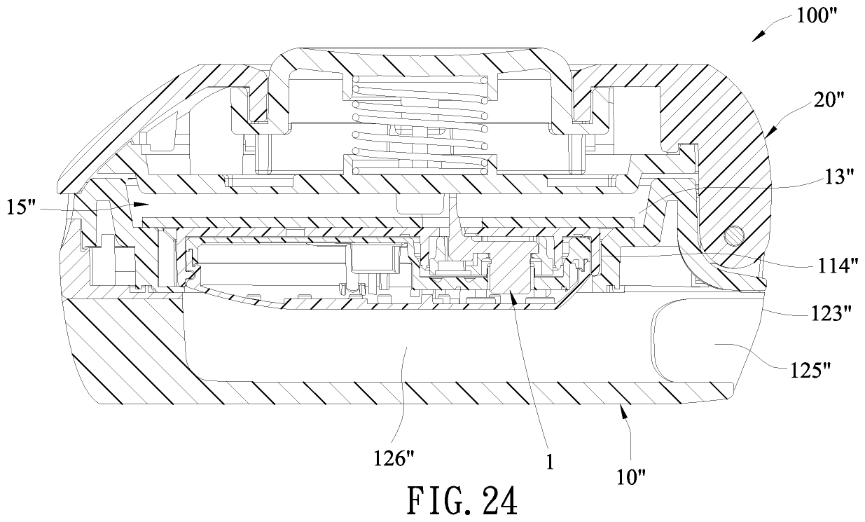

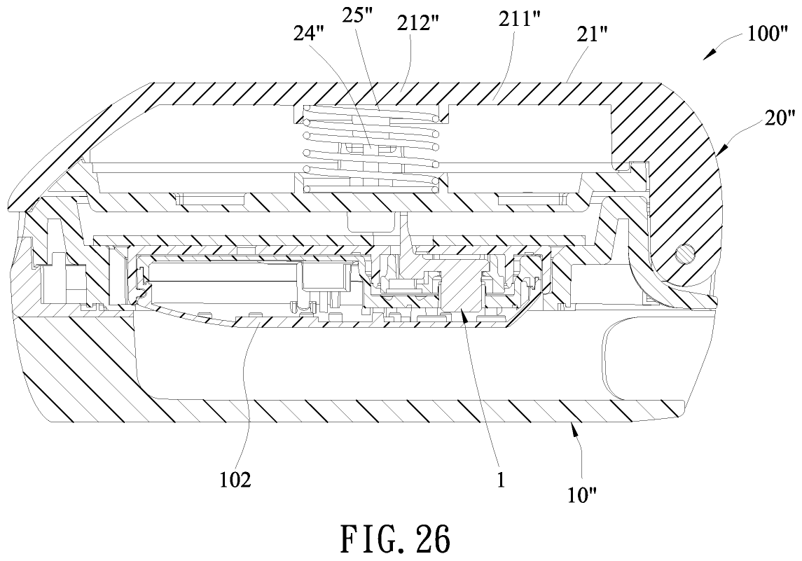

Referring to FIG. 26, an accessory box 100″ of a third embodiment according to the present disclosure is shown. In this embodiment, the cover body 20″ does not include the button 23 shown in FIG. 24, includes only one pushing member 24″, and the upper lid 21″ includes a top wall 211′ having at least one soft portion 212″ that is resiliently deformable and that is made of, e.g., silicon through a two-material injection molding process. The pushing member 24″ is surrounded by the biasing member 25″, is connected to and driven by the soft portion 212″ to move so the pushing member 24″ is moved in the first direction (I) to push the transmitter 102.

In summary, each of the dissembling accessories 100, 100′ and the accessory boxes 100″ of the present disclosure has a simple structure, is easy to manufacture, and is convenient to disassemble the transmitter 102 from the sensor kit of the physiological signal monitoring device 1 for reuse. Thus, the cost for using a continuous glucose monitoring (CGM) system in a long term may be reduced, and the present disclosure is beneficial to the environment and corporate governance for social responsibility.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A disassembling accessory adapted for disassembling a physiological signal monitoring device, the physiological signal monitoring device including a bottom seat that has at least one through hole, a sensor that is mounted to the bottom seat, and a transmitter that is removably mounted to and covering the bottom seat, said disassembling accessory comprising:
a base seat that has an accommodating space; and
a cover body that is connected to one side of said base seat, that is operable to move relative to said base seat between a covering state and an open state, and that has a button and at least one pushing member linked to said button;
wherein, when said cover body is in the open state, said accommodating space is adapted to receive the physiological signal monitoring device therein with the transmitter facing said accommodating space;
wherein, when said cover body is in the covering state, said button is operable to drive said at least one pushing member to move in a first direction such that said at least one pushing member is adapted to extend through the at least one through hole to push the transmitter for disassembling the transmitter from the bottom seat.

2. The disassembling accessory as claimed in claim 1, wherein:
the bottom seat includes
a bottom plate that is disposed to be mounted on a skin surface of a host, and
at least one first coupling structure that is disposed on a top surface of the bottom plate, the at least one through hole being formed through the bottom plate and disposed adjacent to the at least one first coupling structure; and
the transmitter includes
a bottom casing that faces the top surface of the bottom plate of the bottom seat, and
at least one second coupling structure disposed on the bottom casing and removably engaging the first coupling structure when the transmitter covers the bottom seat with the bottom casing facing the top surface of the bottom plate of the bottom seat,
wherein said at least one pushing member is adapted to extend through the at least one through hole of the bottom seat to push the bottom casing of the transmitter to disengage the at least one first coupling structure from the at least one second coupling structure to thereby disassembling the transmitter from the bottom seat.

3. The disassembling accessory as claimed in claim 1, wherein:
said accommodating space has a first receiving space and a second receiving space that is in spatial communication with said first receiving space;
when said cover body is in the open state, said first receiving space is adapted to receive the physiological signal monitoring device therein with the transmitter facing said first receiving space; and
when said cover body is in the covering state, said button is operable to drive said at least one pushing member to move in the first direction such that said at least one pushing member is adapted to extend through the at least one through hole to push the transmitter so that the transmitter is disassembled from the bottom seat and enters said second receiving space.

4. The disassembling accessory as claimed in claim 3, wherein said first receiving space and said second receiving space are arranged in the first direction and are proximate to each other.

5. The disassembling accessory as claimed in claim 3, wherein said base seat further has a first opening in spatial communication with said second receiving space and an external environment.

6. The disassembling accessory as claimed in claim 5, wherein:
said base seat includes a pair of side walls, a front wall connected to said side walls, a rear wall connected to said side walls and opposite to said front wall, and a bottom wall connected to said side walls, said front wall, and said rear wall;
said side walls, said front wall, said rear wall, and said bottom wall cooperatively define said second receiving space; and
said first opening is formed in said bottom wall.

7. The disassembling accessory as claimed in claim 5, wherein:
said base seat includes a pair of side walls, a front wall connected to said side walls, a rear wall connected to said side walls and opposite to said front wall, and a bottom wall connected to said side walls, said front wall, and said rear wall;
said side walls, said front wall, said rear wall, and said bottom wall cooperatively define said second receiving space;
said base seat further has a second opening that is formed in one of said front wall and said rear wall and that is in spatial communication with said second receiving space and the external environment; and
said first opening is formed in said bottom wall.

8. The disassembling accessory as claimed in claim 7, wherein:
said first opening has an opening size that is adapted to be smaller than a base wall area of a base wall of the transmitter;
said second opening is adapted for passage of the transmitter therethrough to exit from said second receiving space to the external environment when the transmitter is disassembled from the bottom seat; and
when the transmitter is disassembled from the bottom seat and falls into said second receiving space, the bottom seat and the sensor is movable out of said base seat by an external force that is applied via said first opening.

9. The disassembling accessory as claimed in claim 5, wherein:
said base seat includes a pair of side walls, a front wall connected to said side walls, a rear wall connected to said side walls and opposite to said front wall, and a bottom wall connected to said side walls, said front wall, and said rear wall;
said side walls, said front wall, said rear wall, and said bottom wall cooperate with each other to define said second receiving space; and
said first opening is formed in one of said front wall and said rear wall.

10. The disassembling accessory as claimed in claim 3, wherein:
said base seat further has a limiting structure disposed adjacent to said first receiving space;
when said cover body is in the open state, said first receiving space is adapted to receive the physiological signal monitoring device therein with the transmitter facing said first receiving space, said limiting structure is adapted to limit the bottom seat of the physiological signal monitoring device in said first receiving space, and to consequently position the transmitter to be adjacent to said second receiving space.

11. The disassembling accessory as claimed in claim 3, wherein:
said disassembling accessory further includes a pivot shaft;
said base seat includes
an upper housing defining said first receiving space, and
a lower housing defining said second receiving space, and connected pivotably to said upper housing via said pivot shaft; and
said lower housing is pivotable relative to said upper housing from a limiting state to a non-limiting state where removal of the transmitter from said second receiving space is permitted.

12. The disassembling accessory as claimed in claim 1, wherein:
said cover body has an upper lid, a lower lid connected to said upper lid, and an inner space defined by said upper lid and said lower lid;
said button being mounted on said upper lid;
said cover body further includes a biasing member disposed in said inner space, and disposed between and abutting against said lower lid and said button for biasing said button in a direction away from said lower lid; and
said at least one pushing member is disposed in said inner space and is driven by said button to move, such that said at least one pushing member is driven to extend outwardly of said lower lid.

13. The disassembling accessory as claimed in claim 1, further comprising a pivot shaft, said cover body being pivotably connected to said base seat via said pivot shaft.

14. The disassembling accessory as claimed in claim 13, further comprising a rotation limiting member mounted to said base seat at a position that is adjacent to said pivot shaft and disposed for positioning said cover body in the open state.

15. A disassembling accessory adapted for disassembling a physiological signal monitoring device, the physiological signal monitoring device including a sensor kit that has at least one through hole at a bottom portion thereof, and a transmitter that is removably coupled to the sensor kit, said disassembling accessory comprising:
a base seat that has an accommodating space and that is adapted for accommodating the physiological signal monitoring device; and
a cover body that is connected to one side of said base seat, that is operable to move relative to said base seat between a covering state and an open state, and that has at least one pushing member;
wherein, when said cover body is in the open state, said accommodating space is adapted to receive the physiological signal monitoring device therein with the transmitter facing said accommodating space;
wherein, when said cover body is in the covering state, said at least one pushing member is operable to move in a first direction such that said at least one pushing member is adapted to extend through the at least one through hole to push the transmitter for disassembling the transmitter from the sensor kit.

16. The disassembling accessory as claimed in claim 15, the sensor kit including a bottom seat that is to be mounted on a skin surface of a host and that is formed with the at least one through hole, and a sensor that is mounted to the bottom seat and that is at least partially disposed between the transmitter and the bottom seat when the transmitter covers the bottom seat, wherein:
said at least one pushing member is adapted to extend through the at least one through hole of the bottom seat in the first direction to push the transmitter such that the transmitter is disassembled from the bottom seat and removed from said base seat in a second direction different from the first direction.

17. The disassembling accessory as claimed in claim 15, wherein said cover body includes a top wall having at least one soft portion that is operable to drive said at least one pushing member to move in the first direction such that said at least one pushing member is adapted to extend through the through hole to push the transmitter for disassembling the transmitter from the sensor kit.

18. The disassembling accessory as claimed in claim 15, wherein:
said cover body has an upper lid, a lower lid connected to said upper lid, and an inner space defined by said upper lid and said lower lid;
said cover body further has
a soft portion formed on said upper lid and being resiliently deformable, and
a biasing member disposed in said inner space, and disposed between and abutting against said lower lid and said upper lid for restoring said soft portion; and
said at least one pushing member is disposed in said inner space and is driven by said soft portion to move in the first direction, such that said at least one pushing member is adapted to extend outwardly of said lower lid through the at least one through hole to push the transmitter for disassembling the transmitter from the sensor kit.

19. The disassembling accessory as claimed in claim 15, wherein:
said base seat further has a limiting structure disposed in said accommodating space;
said accommodating space is adapted to receive the physiological signal monitoring device therein with the transmitter facing said accommodating space, said limiting structure being adapted to limit the sensor kit in a portion of said accommodating space; and
when said pushing member is driven to move for pushing the transmitter to be disassembled from the sensor kit, the transmitter moves to another portion of said accommodating space after being disassembled from the sensor kit to be removed from said base seat.

20. An accessory box adapted for disassembling a physiological signal monitoring device, the physiological signal monitoring device including a sensor kit that has at least one through hole at a bottom portion thereof, and a transmitter removably coupled to the sensor kit, said accessory box comprising:
a base seat that has
a disassembling unit having an accommodating space, and
a charging unit disposed adjacent to said disassembling unit and formed with a charging slot; and
a cover body that is connected to one side of said base seat, that is operable to move relative to said base seat between a covering state and an open state, and that has at least one pushing member disposed adjacent to said disassembling accessory when said cover body is in the covering state;
wherein, when said cover body is in the open state, said accommodating space is adapted to receive the physiological signal monitoring device therein with the transmitter facing said accommodating space;
wherein, when said cover body is in the covering state, said at least one pushing member is operable to move in a first direction such that said at least one pushing member is adapted to extend through the at least one through hole to push the transmitter for disassembling the transmitter from the sensor kit; and
wherein said charging slot of said charging unit is adapted to receive the transmitter that is disassembled from the sensor kit 101+103) for charging a battery of the transmitter.

21. The accessory box as claimed in claim 20, wherein said cover body includes a top wall having at least one soft portion that is disposed adjacent to said disassembling unit when said cover body is in the covering state, and that is operable to drive said at least one pushing member to move in the first direction such that said at least one pushing member is adapted to extend through the through hole to push the transmitter for disassembling the transmitter from the sensor kit.

22. The accessory box as claimed in claim 20, wherein said cover body includes a button that is operable to, when said cover body is in the covering state, drive said at least one pushing member to extend through the at least one through hole of the sensor kit in the first direction to push the transmitter such that the transmitter is disassembled from the sensor kit to thereby allow removal of the transmitter from said accommodating space.

23. The accessory box as claimed in claim 20, wherein:
said accommodating space has a first receiving space and a second receiving space in spatial communication with said first receiving space;
when said cover body is in the open state, said first receiving space is adapted to receive the physiological signal monitoring device therein with the transmitter facing said first receiving space; and
when said cover body is in the covering state, said at least one pushing member is operable to move in the first direction such that said at least one pushing member is adapted to extend through the at least one through hole to push the transmitter so that the transmitter is disassembled from the sensor kit and enters said second receiving space.

24. The accessory box as claimed in claim 20, wherein:
said base seat includes a pair of side walls, a front wall connected to said side walls, a rear wall connected to said side walls and opposite to said front wall, and a bottom wall connected to said side walls, said front wall, and said rear wall;
said side walls, said front wall, said rear wall, and said bottom wall cooperatively define said second receiving space; and
said base seat further has a first opening that is formed in one of said front wall and said rear wall and that is in spatial communication with said second receiving space and an external environment.

25. The accessory box as claimed in claim 20, further comprising an indicating unit that is disposed on an outer surface of said base seat, that is electrically connected to said charging unit, and that is configured to indicate a charging progress of the transmitter.

26. A method for disassembling a physiological signal monitoring device, the physiological signal monitoring device including a sensor kit that is formed with at least one through hole at a bottom portion thereof, and a transmitter that is removably coupled to the sensor kit, the method comprising the steps of:
providing a disassembling accessory that includes
a base seat having an accommodating space for receiving the physiological signal monitoring device therein, and
a cover body connected to one side of the base seat, movable relative to the base seat between a covering state and an open state, and including at least one pushing member;
uncovering the cover body so that the cover body is in the open state relative to the base seat;
placing the physiological signal monitoring device into the accommodating space with the transmitter facing the accommodating space;
covering the cover body so that the cover body is in the covering state relative to the base seat;
disassembling the transmitter from the sensor kit by driving the at least one pushing member to move in a first direction, to extend through the at least one through hole, and to push the transmitter; and
retrieving the transmitter by removing the transmitter from the base seat.

27. The method as claimed in claim 26, the cover body further including a button that is operable to drive the pushing member to move, wherein the step of disassembling the transmitter from the sensor kit further includes:
pushing the button in the first direction to move the at least one pushing member in the first direction, to extend through the at least one through hole, and to push the transmitter.

28. The method as claimed in claim 26, the base seat further having a limiting structure, wherein:
the step of placing the physiological signal monitoring device into the accommodating space further includes limiting the sensor kit by the limiting structure so the physiological signal monitoring device is received in a portion of the accommodating space; and
upon the step of disassembling the transmitter from the sensor kit, moving the transmitter to another portion of the accommodating space.

29. The method as claimed in claim 28, the another portion of the accommodating space having a first opening that is in spatial communication with an external environment, wherein the step of retrieving the transmitter further includes:
removing the transmitter away from the base seat through the first opening.
